(19) United States
(12) Patent Application Publication
(10) Pub. No.: US 2025/0144298 A1
(43) Pub. Date: May 8, 2025

(54) AUTO-INJECTORS AND METHODS FOR ASSEMBLING AND USING THEM

(71) Applicant: ALTAVIZ, LLC, Irvine, CA (US)

(72) Inventors: Matthew McCawley, Grant, FL (US); Jack R. Auld, Laguna Niguel, CA (US); Eric Anderfaas, Westminster, CA (US); John C. Dunne, JR., Costa Mesa, CA (US)

(21) Appl. No.: 18/884,006

(22) Filed: Sep. 12, 2024

Related U.S. Application Data
(60) Provisional application No. 63/538,012, filed on Sep. 12, 2023.

(51) Int. Cl. A61M 5/20; A61M 5/31; A61M 5/315

(52) U.S. Cl. CPC A61M 5/2046; A61M 5/3135; A61M 5/31515; A61M 5/31578; A61M 2005/2013; A61M 2005/208; A61M 2202/0007; A61M 2207/10

(57) ABSTRACT

A method for assembling an auto-injector to minimize a gap between a plunger rod and syringe stopper is provided. A rear housing is provided that includes a gas canister and a plunger including a first end within a chamber of the rear housing and a second end extending from a distal end of the rear housing. A forward housing is provided including a syringe loaded into an interior thereof such that a syringe stopper is positioned adjacent a proximal end of the forward housing. The proximal end of the forward housing is aligned with the distal end of the rear housing such that the second end of the plunger is adjacent the stopper, and the housings are directed towards one another, the stopper causing the plunger to retract proximally within the rear housing to a proximal position to minimize a gap between the plunger and stopper.

### RELATED APPLICATION DATA

The present application claims benefit of co-pending U.S. provisional application Ser. No. 63/538,012, filed Sep. 12, 2023, the entire disclosure of which is expressly incorporated by reference herein.

### TECHNICAL FIELD

The present application relates generally to devices and methods for delivering agents into a subject's body and, more particularly, to auto-injectors and/or gas-powered drive systems for injection devices, and to methods for manufacturing, assembling, and using such devices.

### BACKGROUND

There are many applications involving delivery of a medicament or other agent subcutaneously, intramuscularly, or otherwise into a patient's body. For example, auto-injectors are available that include a predetermined dose of the agent that may be delivered automatically into the patient's body, e.g., after placement against the patient's skin and activation. Generally, such auto-injectors are spring-loaded syringes that are activated to release the spring, which generates sufficient force to penetrate the skin with a needle and deliver the dose within the syringe. For viscous fluids, the forces required to develop fluid flow can be higher than spring-powered systems can provide. When springs can be used, they must generate a relatively high force that requires springs of high mass. Consequently, such auto-injectors may make substantial noise, create pressure spikes in the syringe leading to glass breakage, vibrate, and/or may drive the needle forcefully into the patient's skin, which may cause pain and/or may startle the user, particularly when the patient is administering the injection themselves.

For example, with higher viscosity and larger volumes, standard spring-powered autoinjectors must employ bigger springs to deliver the therapies in a reasonable time and result in increased impact forces, leading to syringe breakage in conventional pre-filled syringes (PFS). Additionally, companies are being forced to gravitate to the use of high-performance polymer syringes as a substitute to glass syringes due to the need for increased toughness. Doing so comes with significant costs and regulatory challenges as this restriction presents a major obstacle in the development of novel medications. The significant forces generated by the larger springs necessitate careful consideration of the delivery techniques for these high-volume/high-viscosity medicines. Additionally, the loud impact noise generated during delivery can negatively affect the user experience, particularly for patients with a fear of injections.

In addition, spring driven auto-injectors generally rely on a preloaded plunger rod to apply the necessary force to dispense the contents in the syringe. As such, as shown in FIG. 7, a gap “GAP” generally exists between the syringe stopper S and the plunger rod P of such auto-injectors to account for manufacturing tolerance and syringe fill variation. This gap can allow rapid acceleration and high impact forces to be exerted on the brittle, glass syringe from the plunger rod.

Therefore, improved devices and methods for delivering agents into a patient's body would be useful.

### SUMMARY

The present application relates generally to devices and methods for delivering agents into a subject's body and, more particularly, to auto-injectors and/or gas-powered drive systems for injection devices, and to methods for manufacturing, assembling, and using such devices.

In accordance with one example, a method is provided for assembling an injector device that includes providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end; providing a rear housing comprising proximal and distal ends, a source of pressurized gas within the rear housing, and a plunger including a first end within a chamber of the rear housing such that gas released from the source travels to the chamber to direct the plunger distally; with a second end of the plunger extending from the distal end of the rear chamber, aligning the proximal end of the forward housing with the distal end of the rear housing such that the second end of the plunger is adjacent the stopper; and directing the proximal end of the forward housing towards the distal end of the rear housing, thereby causing the plunger to retract proximally within the rear housing to a proximal position.

In accordance with another example, a method is provided for assembling an injector device that includes providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end; providing a rear housing comprising proximal and distal ends, a source of pressurized gas within the first chamber, and a plunger including a first end within a chamber of the rear housing such that gas released from the source travels to the chamber to direct the plunger distally; with a second end of the plunger extending from the distal end of the rear housing in a distal-most position, aligning the proximal end of the forward housing with the distal end of the rear housing such that the second end of the plunger is adjacent the stopper; directing the proximal end of the forward housing towards the distal end of the rear housing, thereby causing the plunger to retract proximally within the rear housing to a proximal position; and coupling one or more connectors to secure the forward and rear housings together, the plunger being positioned in a proximal-most position.

In accordance with yet another example, a method is provided for assembling an injector device that includes providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end; providing a rear housing comprising proximal and distal ends, a source of pressurized gas within the rear housing, and a plunger including a proximal end within a chamber of the rear housing such that gas released from the source travels to the chamber to direct the plunger distally; and with the plunger in an initial position proximal to a distal-most position within the chamber, coupling the proximal end of the forward housing to the distal end of the rear housing such that a distal end of the plunger is spaced apart proximally from the stopper, e.g., by a predetermined gap distance.

In accordance with still another example, a device is for delivering one or more agents into a subject's body that includes an outer housing comprising a proximal portion terminating in a proximal end and a distal portion terminating in an open distal end; a syringe comprising a barrel containing one or more agents, a needle extending distally from the barrel such that a tip of the needle is disposed within the distal portion adjacent the distal end, and a stopper within a proximal end of the barrel; an inner housing within the outer housing comprising one or more chambers therein; an activation cap on the distal end of the housing such that a contact surface is disposed distal to the outer housing distal end; a source of pressurized gas within the inner housing; a release cap within the outer housing carrying an opener pin located adjacent the source of pressurized gas; and a plunger comprising a proximal end within a chamber of the one or more chambers and a distal end positioned adjacent and spaced apart proximally from the stopper by a gap distance. The activation cap is operably coupled to the release cap and movable axially relative to the outer housing such that, when the contact surface of the activation cap is pressed against a subject's skin, the activation cap is configured to move proximally to direct the release cap to cause the opener pin to open the source of pressurized gas to release the pressurized gas into the chamber such that the pressurized gas advances the plunger to direct the distal end of the piston distally into contact with the stopper to advance the stopper to deliver the one or more agents through the needle.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 is a perspective view of an exemplary auto-injector device including a forward and rear housing, and a safety cap.

FIGS. 2A and 2B are longitudinal cross-sections of the device of FIG. 1 taken along orthogonal planes 2A-2A and 2B-2B, respectively, showing internal components of the device including a syringe carrying one or more agents.

FIGS. 3A and 3B are details of the distal end of the device of FIGS. 1-2B before use, showing a safety cap covering an activation cap and needle of the device.

FIGS. 4A-4C are cross-sections of the device of FIGS. 1-2B, showing activation of the device when the activation cap is displaced proximally when the device is pressed against a subject's skin to puncture the skin with the needle, the activation cap causing a syringe spacer and release cap to also displace proximally to release a carriage carrying an opener pin, causing the opener pin to advance distally to open a gas canister to release pressurized gas into a flow path within the device.

FIGS. 5A-5C are details of the proximal end of the device as shown in FIGS. 4A-4C, respectively.

FIGS. 6A and 6B are cross-sections of the device of FIGS. 1-6 after the pressurized gas is released, showing the pressurized gas advancing a plunger within the device to deliver one or more agents within the syringe through the needle, and advancing the activation cap when the needle is withdrawn from the subject's skin.

FIG. 6C is a detail of the device as shown in FIG. 6B, showing a tab locking the activation cap after being advanced.

FIG. 7 is a cross-sectional schematic of a conventional spring-driven auto-injector showing a nominal gap between a plunger rod and syringe stopper of the auto-injector.

FIGS. 8A and 8B are cross-sectional views of another auto-injector including a gap between a plunger rod and syringe stopper of the auto-injector after assembly.

FIGS. 9A-9C show an exemplary method for assembling an auto-injector to eliminate a gap between a plunger rod and syringe stopper of the auto-injector.

FIG. 10 is a graph showing a simulation comparing impact forces applied to a syringe stopper by a plunger rod of a spring-powered auto-injector and a gas-powered auto-injector.

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

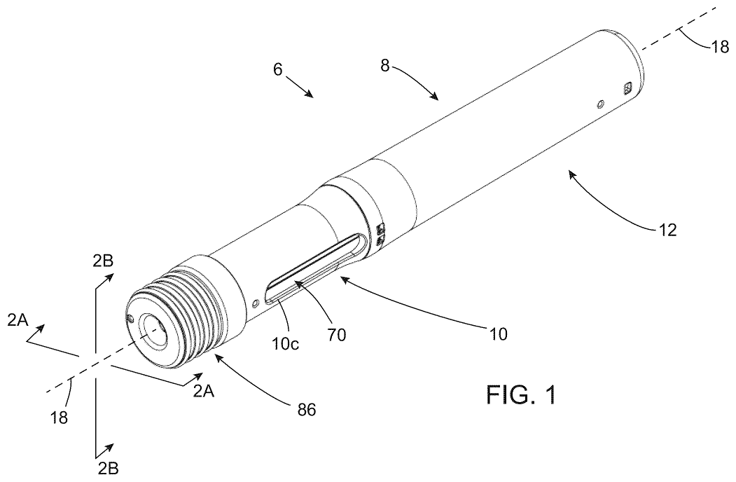

Turning to the drawings, FIG. 1 shows an example of an auto-injector device 6 that includes an outer housing 8 including a forward housing 10 carrying a syringe 70 containing one or more agents within an agent chamber 73, and a rear housing 12 including a drive assembly for automatically delivering the agent(s) from the syringe 70 when the device 6 is activated. As shown, a safety cap 86 is provided on the forward housing 10 to prevent premature activation of the device 6, which may be removed immediately before an injection, as described further elsewhere herein. One or more internal components of the device 6 may move axially, i.e., proximally and/or distally relative to the longitudinal axis 18 when the device 6 is activated, as described further elsewhere herein.

In one example, the syringe 70 may be a pre-filled syringe, e.g., formed from glass, plastic, and the like, filled with a predetermined volume of agent, e.g., corresponding to a single dose for a patient. Alternatively, the agent chamber and needle may be integrated into the forward housing 10 if desired (not shown). In a further alternative, the syringe 70 (or integral agent chamber) may include a distal port (not shown) without a needle, such that a separate needle (also not shown) may be coupled to the port, e.g., using a Luer fitting, mating threads, and/or other cooperating connectors, immediately before an injection or otherwise as desired. As used herein, “agent” may include one or more flowable therapeutic and/or diagnostic compounds, medicaments, or materials, e.g., in liquid or gaseous form, in solution or suspension, and the like, such as viscous fluids.

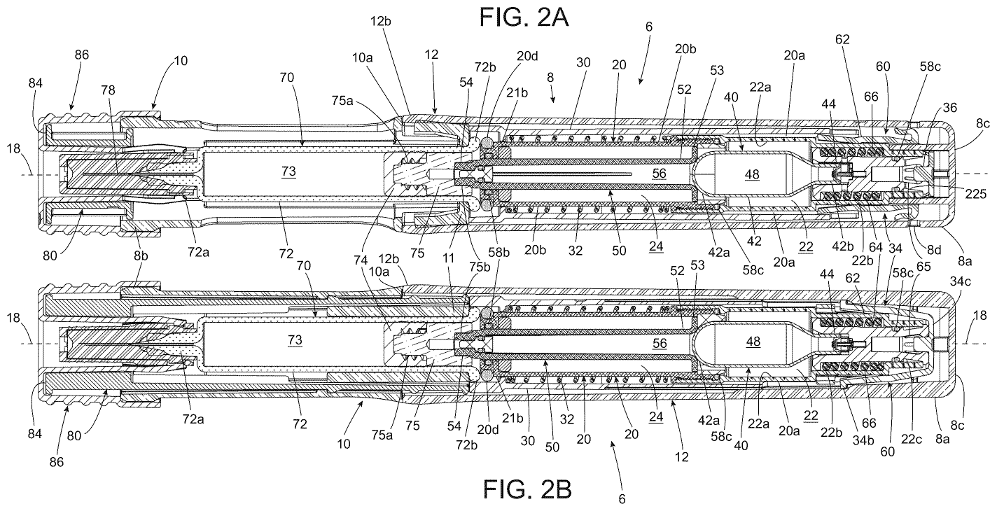

With additional reference to FIGS. 2A and 2B, the syringe 70 includes a barrel 72 containing the agent(s) within agent chamber 73, a needle 78 extending from a distal end 72a of the barrel 72, and a piston or stopper 74 slidable within a proximal end 72b of the barrel 72 for directing the agent(s) through the needle 78 into the subject's body (not shown). The device 6 also includes a gas canister or other source of pressurized gas 40 to power the device 6, e.g., to advance a plunger 50 coupled to the stopper 74 to deliver the agent(s). The device 6 also includes an activation cap 80 and an opener mechanism 60 for opening the canister 40 to release pressurized gas within the canister 40 into a set of chambers of the device 6. For example, an inner housing 20 may be mounted within the outer housing 8 that includes a first or proximal chamber 22, e.g., within which the gas canister 40 is mounted, and a second or distal chamber 24, e.g., in which the plunger 50 is slidably received.

Generally, the device 6 is configured such that, when a contact surface 84 of the activation cap 80 is pressed against a subject's skin to insert the needle 78, the activation cap 80 retracts proximally into the outer housing 8 and automatically activates the opener mechanism 60 to release the pressurized gas from the canister 40 into the first and second chambers 22, 24, which generates a distal force to advance the plunger 50 to deliver the agent(s) from the syringe 70 into the subject. After the injection is completed and the device 6 is withdrawn away from the skin, the activation cap 80 may automatically advance to cover the needle 78.

For example, a spacer 30 may be provided that extends between the activation cap 80 and a release cap 34 adjacent the opener mechanism 60, e.g., such that proximal movement of the activation cap 80 is translated by the spacer 30 to the direct the release cap 34 proximally within the rear housing 12, which releases the opener mechanism 60, as described further elsewhere herein. The spacer 30 may be biased distally, e.g., by spring 32, which may, in turn, bias the activation cap 80 distally after completing the injection, also as described further elsewhere herein.

Generally, the outer housing 8 includes a proximal end 8a, e.g., provided on the rear housing 12, an open distal end 8b, e.g., provided on the forward housing 10, and an inner wall extending therebetween to enclose the interior of the device 6. The outer surface between the proximal and distal ends 8a, 8b may be sized and/or shaped to facilitate manipulation of the device 6, e.g., to facilitate placing and pressing the activation cap 80 against a subject's skin to activate the device 6 and inject the agent(s). For example, the outer surface may have a generally cylindrical shape optionally including one or more textures or grip features to facilitate an operator (the subject themselves or a third party) holding the device 6 in one hand and pressing the activation cap 80 against the subject's skin, as described further elsewhere herein. As shown, the proximal end 8a of the housing 8 may include a wall 8c enclosing the proximal end 8a and, optionally, one or more pockets or other features 8d for locking the release cap 34, as described further elsewhere herein.

The outer housing 8 may be formed from multiple, separate components, e.g., clamshell halves, e.g., formed from metal, such as stainless steel, aluminum, and the like, plastic, and/or composite material, by one or more of cold drawing, molding, casting, machining, and the like, that are substantially permanently attached together, e.g., by one or more of welding, soldering, fusing, bonding with adhesive, interference fit, and the like. As shown, the outer housing 8 is formed as separate forward and rear housings 10, 12, which may be assembled separately with respective internal components, and then coupled together, e.g., after loading a syringe 70 into the forward housing 10.

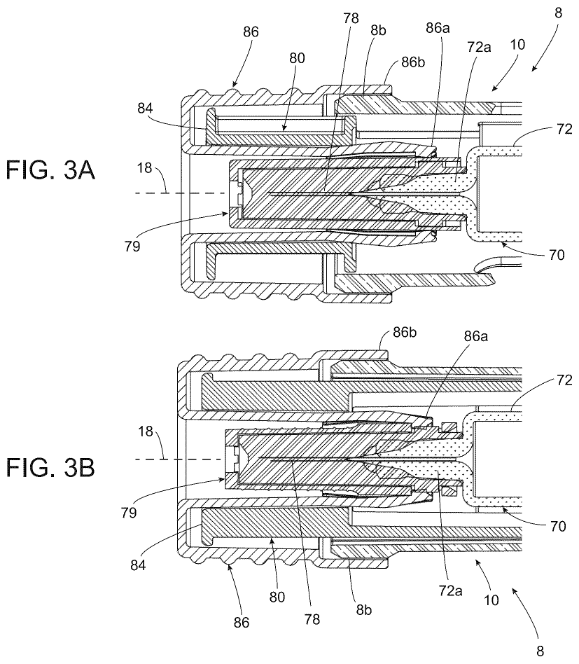

For example, during manufacturing, the forward housing 10 may be assembled with the activation cap 80 and safety cap 86, and a syringe 70 may be loaded into an interior of the forward housing 10, e.g., during manufacturing or during assembly of the device 6 any time before performing an injection. Optionally, as shown in FIGS. 3A and 3B, the syringe 70 may include a rigid needle shield 79, e.g. formed from conventional materials, that may be removably secured to the distal end 72a of the barrel 72 to protect the needle and prevent accidental sticks. The safety cap 86 may include features 86a that engage the needle shield 79 such that, when the safety cap 86 is removed, the needle shield 79 is also removed. For example, as shown, the proximal end of the safety cap 86 may include one or more fingers 86a that fall into one or more corresponding cutouts, recesses, or other features on the needle shield 79 when the syringe 70 is inserted into the forward housing 10, thereby coupling the needle shield 79 to the safety cap 86, as described elsewhere herein.

The rear housing 12 may be assembled with the inner housing 20, the spacer 30, the release cap 34, the gas canister 40, the plunger 50, and the opener mechanism 60 (and any other incidental components), and the rear housing 12 may be coupled to the forward housing 10 at any time. For example, during manufacturing, a syringe 70 may be loaded into the interior of the forward housing 10, and the assembled rear housing 12 may be immediately coupled to the forward housing 10. When the rear housing 12 is attached to the forward housing 10, a distal end 54 of the plunger 50 may be coupled to the stopper 74 within the syringe 70, e.g., such that distal movement of the plunger 50 is translated to the stopper 74, as described further elsewhere herein.

As shown in FIGS. 2A and 2B, the outer housing 8 may include a mount 11, e.g., on one of the forward and rear housings 10, 12, for guiding and/or securing the syringe 70 during loading. Optionally, the proximal end 72b of the barrel 72 may include one or more flanges, e.g., a radial flange or a pair of opposing flanges, that may abut and/or be received within the mount 11 to secure the syringe 70 relative to the forward housing 10. In addition or alternatively, one or more detents, ridges, or other features (not shown) may be provided on the forward housing 10 for securing the syringe 70. Optionally, the forward housing 10 may include one or more windows 10c in the sidewall, which may facilitate observing the syringe 70 during an injection, e.g., to allow visual confirmation when the stopper 74 is fully advanced.

Optionally, the device 6 may include a syringe spacer or adapter 75 that may provide an interface between a distal end 54 of the plunger 50 and the stopper 74, e.g., to provide connectors therebetween and/or ensure proper spacing such that the stopper 74 is advanced in conjunction with the plunger 50. For example, different length spacers 75 may be provided to allow different length syringes to be loaded into the forward housing 10 while properly positioning the needle 78 adjacent the distal end 8b of the outer housing 8 and allowing the plunger 50 to be properly coupled to the stopper 74.

During manufacturing or assembly, a syringe 70 may be selected that may be inserted into the housing 8, e.g., through the opening in the distal end 8b and coupled to the distal end 16 of the drive assembly 12. A syringe spacer 75 may be selected and its distal end 75a may be coupled to the stopper 74, e.g., by one or more cooperating threads, as shown, one or more detents or other connectors, and the like. The distal end 54 of the plunger 50 may be inserted into a recess 75b in the syringe spacer 75 when the rear housing 12 is coupled to the forward housing 10. Optionally, the distal end 54 of the plunger 50 and the recess 75b may include cooperating locking features, e.g., one or more threads, detents, and the like (not shown) or the distal end 54 may simply be inserted into the recess 75b to coupled subsequent distal movement of the stopper 74 to the plunger 50, as described further elsewhere herein.

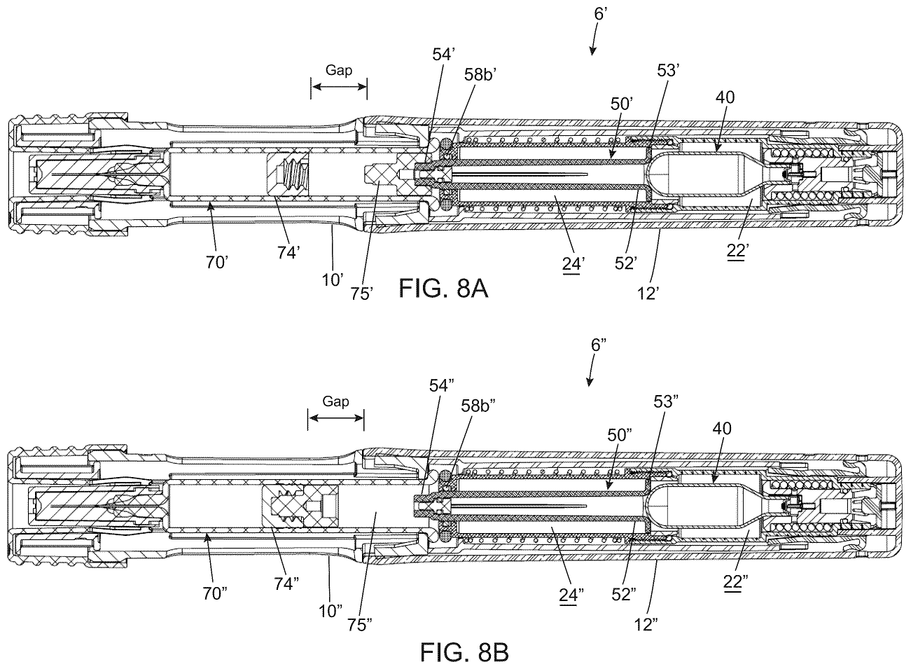

Alternatively, as shown in FIGS. 8A and 8B, the auto-injector devices may be assembled similar to any of the other examples herein, but without a spacer such a gap is provided between the plunger 50′ and the stopper 74.′ For example, as shown in FIG. 8A, the device 6′ may be assembled such that the plunger 50′ is positioned in its proximal position, resulting in the distal end 54′ of the plunger 50′ being spaced proximally from the stopper 74′ by a gap distance. In the example shown in FIG. 8A, a piston member 75′ may be provided on the distal end 54′ of the plunger 50′ such that the gap distance is provided between the tip of the piston member 75′ and the back of the stopper 74′. The tip of the piston member 75′ may be sized and/or configured to be seated in or otherwise engage the stopper 74′ in the syringe 70′ when the device 6′ is activated.

Alternatively, in the example shown in FIG. 8B, the device 6″ may be provided with a piston member 75″ on the stopper 74″ such that the gap distance is provide between the distal end 54″ of the plunger 50″ and the back of the piston member 75.″ The distal end 54″ of the plunger 50″ may be sized and/or configured to be seated in or otherwise engage the piston member 75″ when the device 6″ is activated. Otherwise, the devices 6,′ 6″ may be constructed, assembled, and/or used similar to other devices described herein.

In an exemplary method with reference to the device 6′ shown in FIG. 8A (although applicable to other devices herein), the plunger 50′ may be positioned in a position proximal to its distal-most position during assembly of the device 6.′ For example, when the rear housing 12′ is assembled, the plunger 50′ may be positioned in an initial position, e.g., at its proximal-most-position with the flange 53′ on its proximal end 52′ provided at the proximal end of the chamber 24.′ Thereafter, the proximal end 10a′ of the forward housing 10′ may be aligned with and coupled to the distal end 12b′ of the rear housing 12,″ similar to other assembly methods herein, such that the distal end 54′ of the plunger 50′ is spaced apart proximally from the stopper 74′ by the gap distance.

Optionally, the plunger 50′ and/or the second chamber 24′ may include one or more features that prevent distal migration of the plunger 50′from its initial position before the forward housing 10′ is coupled to the rear housing 12.′ For example, a sealing member 58b′ may be provided within the second chamber 24′ that slidably contacts the plunger 50′ but allows the plunger 50′ to move axially. Alternatively, a frictional member (not shown) may be provided on the plunger 50′ and/or within the second chamber 24′ that resists distal movement of the plunger from the initial position until a threshold frictional resistance is overcome (e.g., by pressurized gas released during use and activation of the device 6,′ as described elsewhere herein). For example, when the rear housing 12′ is assembled, the axial position of the plunger 50′ may be adjusted as desired, e.g., to a desired initial axial position proximal to its distal-most position by overcoming the friction, e.g., to position the distal 54′ at a desired location to ensure a gap distance is provided in the assembled device 6.′

Thus, whether a piston member 75′ is provided on the distal 54′ of the plunger 50′ or on the stopper 74″ (as shown in FIG. 8B), the plunger 50,′ 50″ may remain spaced apart from the stopper 74,′ 74.″ This assembly method may ensure that the stopper 74,′ 74″ does not move during assembly of the device 6,′ 6,″ which may otherwise risk ejecting agent(s) from the syringe 70,′ 70″ and/or disturbing the sterile field of the syringe 70,′ 70.″

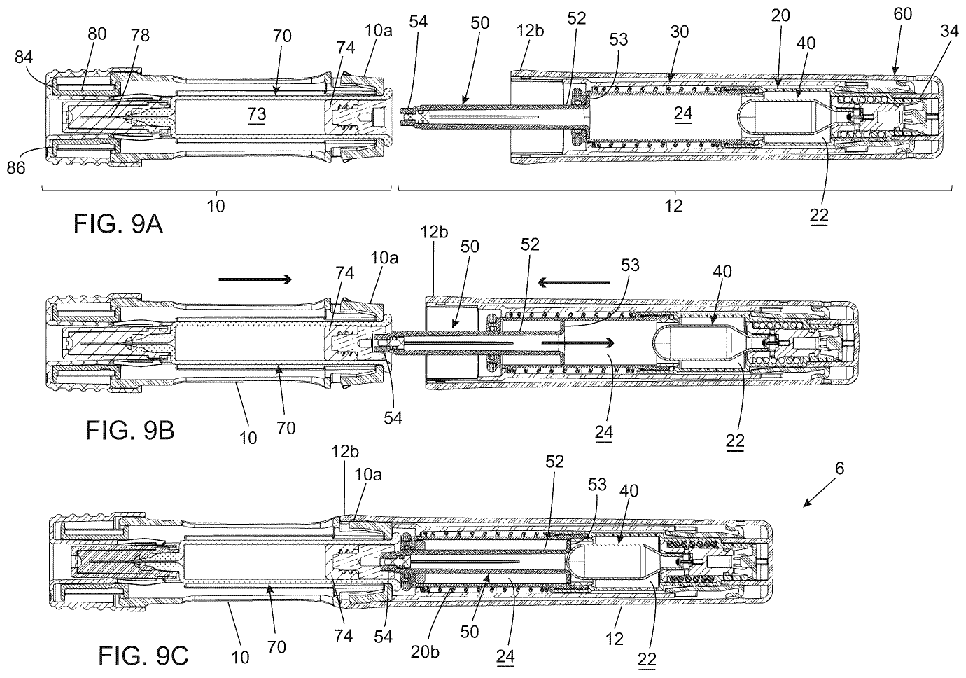

In a further alternative, the device 6 may be assembled such that there is no need for providing a specialized spacer or gap between the plunger 50 and the stopper 74. For example, turning to FIGS. 9A-9C, an exemplary method is shown for assembling an auto-injector device 6, such as any of the devices described herein. Initially, the components of the device 6 may be assembled into the forward housing 10 and the rear housing 12, including the components described elsewhere herein, e.g., during manufacturing and/or assembly by the manufacturer. For example, the forward housing 10 may be manufactured and assembled to include the activation cap 80 and safety cap 86. A syringe 70 including one or more agents may be inserted into the interior of the forward housing 10, e.g., through the proximal end 10a, during manufacturing and/or assembly, e.g., by the manufacturer, or during assembly of the device 6 immediately before performing an injection, e.g., by a physician or other medical personnel.

In addition, the rear housing 12 may be manufactured and assembled, e.g., to include the inner housing 20, the spacer 30, the release cap 34, the gas canister 40, the plunger 50, and the opener mechanism 60 (and any other incidental components), e.g., again during manufacturing and/or assembly by the manufacturer. As shown in FIG. 9A, when the rear housing 12 is fully assembled, the distal end 54 of the plunger 50 may extend from the distal end 12b of the rear housing 12. For example, the plunger 50 may be provided in a distal-most position, e.g., with a flange 53 on the proximal end 52 of the plunger 50 abutting the distal end 21b of the distal cylinder 20b.

With the forward and rear housing 10, 12 individually assembled (and with a syringe 70 loaded into the forward housing 10), as shown in FIG. 9B, the proximal end 10a of the forward housing 10 may be aligned with the distal end 12b of the rear housing 12 such that the second end 54 of the plunger 50 contacts the piston member 75. The proximal end 10a of the forward housing 10 may be directed towards the distal end 12b of the rear housing 12, thereby causing the plunger 50 to retract proximally within the rear housing 12. For example, during this assembly, the stopper 74 does not move relative to the syringe barrel 72 the plunger 50 is retracted proximally by the stopper 74 pushing the plunger 50 proximally as the housings 10, 12 come together. With the first and second chambers 22, 24 within the rear housing 12 sealed, residual air or gas within the second chamber 24 may be compressed as the plunger 50 is retracted, but the resulting pressure may be insufficient to move the stopper 74, unlike the substantial pressure generated when the pressurized gas within the canister 40 is released.

As shown in FIG. 9C, the proximal end 10a of the forward housing 10 may be directed towards the distal end 12b of the rear housing 12 until the ends 10a, 12b are engaged or otherwise coupled together to provide the assembled device 6. For example, the ends 10a, 12b may include one or more connectors, e.g., detents, threads, and the like, that engage to secure the forward and rear housings 12, 12 together.

Consequently, after assembly, as shown in FIG. 9C, the plunger 50 is positioned in a proximal-most position without a substantial gap between the distal end 54 of the plunger 50 and the stopper 74 such that, when pressurized gas is released from the source of pressurized gas 40 when the device 6 is subsequently activated, the pressurized gas advances the plunger 50 towards a distal position and immediately applies a distal force to the stopper 74. This configuration may provide a significant advantage over conventional spring-powered auto-injectors.

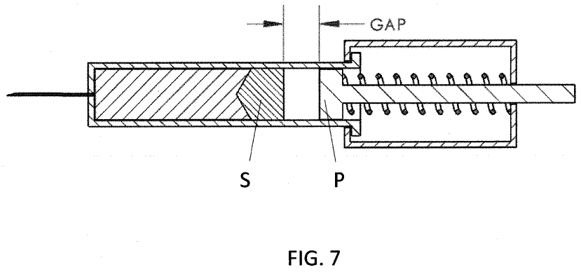

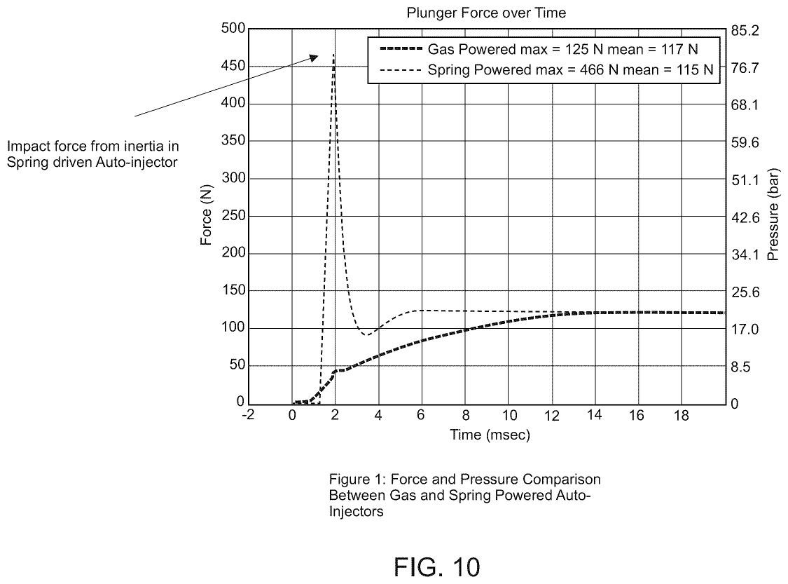

For example, FIG. 10 shows relative impact forces that may be experienced by spring-powered and gas-powered auto-injectors. The lighter line represents the impact forces that may experienced by a stopper during activation of a spring-powered injector. Initially, particularly if there is a gap between the plunger and stopper, e.g., as shown in FIG. 7, the initial inertia from the spring may cause a substantial impact force when the spring causes the plunger to rapidly advance and then contact the stopper. Once the plunger and stopper are engaged, the impact force drops dramatically to a more uniform force as the entire system, i.e., plunger, stopper, and agent(s) within the syringe move. In the example shown, the initial impact force may spike to about 466 Newtons and then drop to about 115 Newtons during delivery of the agent(s). This initial impact force may limit the target deliver force applied during delivery, particularly, if the initial impact force risks breaking or damaging the syringe, thereby limiting the spring that may be selected.

In contrast, the darker line represents the impact forces experienced by a stopper during activation of a gas-powered injector, such as that shown in FIGS. 9A-9C without a gap between the plunger 50 and stopper 74. Because the pressurized gas provides a constant pressure when released, e.g., about 125 Newtons, the resulting impact force on the stopper 74 does not spike but simply ramps up to the pressure of the pressurized gas within the canister 40. Consequently, there is no initial spike that risks breaking or damaging the syringe 70. Even in the case of the devices 6,′ 6″ with an initial gap between the plunger 50,′ 50″ and stopper 74.′ 74,″ the impact forces during activation may still be substantially smaller and more uniform compared to conventional spring-powered auto-injectors.

Although the initial release of pressurized gas may generate a higher force when initially advancing the plunger 50,′ 50″ towards the stopper 74,′ 74,″ there is no substantial spike in impact force before contacting and advancing the stopper 74,′ 74.″ For example, given the low profile and relative lightweight construction of the plunger 50,′ 50,″ the collective mass of the plunger 50,′ 50″ and gas initially released from the canister 40 during activation is relatively low. Thus, the initial impact force when the plunger 50,′ 50″ advances and contacts the stopper 74,′ 74″ more closely approximates the exponential curve shown resulting from the pressure increasing with the chambers as the gas is released from the canister 40. In contrast, when a spring from a spring-powered device is initially released, the large mass and potential energy stored in the compressed spring create a substantial spike in the initial impact force, as shown.

Thus, the devices 6-6″ herein may provide substantially more uniform impact forces than conventional spring-powered devices, particularly if the pressure of the pressurized gas is set to a comparable target pressure for the spring of a spring-powered device. Further, given that there is no substantial spike in initial impact force, the pressure within the gas canister may be increased compared to spring-powered injectors, which may allow more rapid delivery of the agent(s) and/or higher viscosity agents may be delivered using gas-powered injectors.

Returning to FIGS. 1-6, the assembled housings 10, 12 may be packaged, stored, and/or otherwise prepared for subsequent assembly. For example, the housing 10, 12 may be shipped or otherwise provided to a manufacturer of the syringe 70, who may load the syringe 70 and couple the housings 10, 12 together to provide the finished device 6. In a further alternative, the assembled housings 10, 12 may be provided to a hospital, physician, or other medical professional, and final assembly may be completed immediately before performing an injection, which may allow a syringe including a specific agent to be loaded before an injection.

The proximal end 10a of the forward housing 10 and the distal end 12b of the rear housing 12 may include one or more cooperating connectors (not shown) to couple the housings 10, 12 together to provide the fully-assembled device 6. For example, the housings 10, 12 may include one or mating threads, detents, and the like, which may permanently couple the housings 10, 12 together. Alternatively, the connectors may allow the housings 10, 12 to be subsequently separated, e.g., after performing an injection, to remove the syringe 70, e.g., to allow the device 6 to be cleaned and reused with a new syringe.

In addition or alternatively, the housings 10, 12 may be permanently attached together by one or more of an interference fit between the ends 10a, 12b, bonding with adhesive, fusing, sonic welding, and the like. In another alternative, the outer housing 8 may be formed as a single, integral component into which the components of the device 6 may be assembled. In these alternatives or even with a two-part housing, the device 6 may be a single use device, which may be disposed of after a single injection.

In the example shown in FIGS. 2A and 2B, the inner housing 20 includes a proximal cylinder 20a including the first chamber 22 and a distal cylinder 20b including the second chamber 24. Each of the proximal and distal cylinders 20a, 20b may have an elongate cylindrical wall or other cross-sectional shape along their lengths. The proximal and distal cylinders 20a, 20b may be permanently attached together to provide the inner housing 20, e.g., by one or more of cooperating threads, as shown, detents or other connectors, force fit, bonding with adhesive, sonic welding, fusing, and the like. Alternatively, the inner housing 20 may be formed as a single, unitary structure including the first and second chambers 22, 24. The proximal and distal cylinders 20a, 20b may be formed from materials similar to or different from the outer housing 8.

The inner housing 20 may be mounted within the outer housing 8 such the inner housing 20 remains substantially stationary within the outer housing 8. Similarly, the gas canister 40 may be mounted within the inner housing 20, e.g., within the first chamber 22, such that the gas canister 40 also remains substantially stationary relative to the outer housing 8. For example, one or more struts, supports, or other structures (not shown) may be provided on the inner and/or outer housings 20, 8 to secure the inner housing 20 and/or the gas canister 40 relative to the outer housing 8.

In the example shown, the proximal cylinder 20a may include an annular wall surrounding the first chamber 22 that includes a uniform diameter first region 22a within which the gas canister 40 is mounted, and second and third regions 22b, 22c within which the opener mechanism 60 is slidably mounted, as described further elsewhere herein. The distal cylinder 20b may have a length such that a distal end 21b of the distal cylinder 20b may abut the syringe 70 when the device 6 is assembled.

Optionally, the distal end 21b may include one or more features that contact the proximal end 72b of the syringe 70, e.g., an O-ring 20d secured on or around the distal end 21b formed from resilient and/or relatively flexible material. The O-ring 20d may prevent the distal end 21b from damaging the syringe 70, e.g., if the barrel 72 is made from glass.

With continued reference to FIGS. 2A and 2B, the canister 40 includes a body 42 including a first closed end 42a, a second outlet end 42b, and a cap 44 welded or otherwise attached to the outlet end 42b to provide an enclosed cavity 48 filled with a fluid containing pressurized gas, such as carbon dioxide or fluorocarbon gases, compressed to sufficient pressure to least partially liquefy the gas within the cavity 48. Alternatively, fluids containing gases such as argon, nitrogen, helium argon, or other combinations thereof that remain in gaseous form may be stored within the cavity 48. As described elsewhere herein, the pressurized fluid contained within the cavity 48 may be used to generate the forces to operate the device 6, e.g., to inject one or more agents from the syringe 70 into a subject's body.

In one example, the body 42 and cap 44 may be formed from stainless steel or other desired or suitable metal, plastic, or composite material, e.g., formed by one or more of drawing, stamping, machining, casting, molding, and the like. For example, the body 42 may be deep drawn from sheet metal, e.g., a round sheet metal blank of Type 305 stainless steel, using one or more dies and punches (not shown), to form a main barrel region, the enclosed end 42a, an optional tapered shoulder region, and the outlet end 42b defining an opening to which the cap 44 is attached.

As shown, the canister 40 may be oriented with the outlet end 42b proximal to the enclosed end 42a, and the opener mechanism 60 may be provided proximal to the outlet end 44.

In the example shown, the cap 44 may be an enclosed cap including a septum or other weakened region (not shown) that may be opened by the opener mechanism. In this example, the opener mechanism may include an opener pin 62 configured to puncture or preferentially tear the septum, as described elsewhere herein. Additional information regarding canisters that may be used and methods for making them may be found in U.S. Publication No. 2017/0258583, the entire disclosure of which is expressly incorporated by reference herein.

Alternatively, the cap 44 may include other closer mechanisms, such as a ball or other member (not shown) that may be biased or otherwise configured to close an outlet in the cap 44 yet may be directed away from the cap 44, e.g., into the canister 40, by the opener mechanism 60 to open the outlet and release the pressurized gas within the cavity 48. In this alternative, the opener pin 62 may include a tapered tip (not shown) sized to enter the outlet and push the closure away from the outlet.

The plunger 50 may be an elongate rod or other member including a proximal end 52 that is slidably disposed within the second chamber 24, e.g., initially immediately adjacent the first chamber 22, and a distal end 54 coupled to the stopper 74. The plunger 50 is movable from an initial or retracted position (e.g., shown in FIGS. 2A and 2B) to a final or extended position (e.g., shown in FIGS. 7A and 7B), e.g., wherein the distal end 54 extends from the second end 16 of the drive assembly 12 into the agent chamber 73 of the syringe 70.

A flange or other guide member 53 may be provided on the proximal end 52 of the plunger 50 that slidably engages a wall of the second chamber 24. Consequently, when pressurized gas enters the second chamber 24 (via the first chamber 22), the pressure generates a distal force to direct the plunger 50 distally from the initial position towards the final position to advance the stopper 74 and deliver the one or more agents from the agent chamber 73 through the needle 78 into the subject, as described further elsewhere herein.

Optionally, a wall, orifice, or intermediate passage (not shown) may be provided between the first and second chambers 22, 24. The intermediate passage may have a relatively small diameter to provide a restrictor to reduce pressure rise time within the second chamber 24. For example, the intermediate passage may i) slow down the transient flow of gas, slowing the rise of pressure imparted to the plunger 50, e.g., providing a soft-start to the injection, reducing/eliminating pressure shock waves in the fluid to be injected in the syringe and possibly reducing patient pain as the drug injection is gently initiated; and/or ii) slow down the steady state flow of gas, reducing the otherwise pressure imparted to the plunger 50, providing a limiting effect to the flow rate of the drug injected into the patient.

Optionally, as shown, the plunger 50 may also include a plunger chamber 56, e.g., extending from an open proximal end 52 of the plunger 50 to a closed distal end 54. The plunger chamber 56 may taper inwardly from the proximal end 52 to the distal end 54, as shown, or may have a uniform diameter or other cross-sectional shape along its length. Alternatively, the proximal end 52 of the plunger 50 may include a closed wall and/or the plunger 50 may be a solid rod between the proximal and distal ends 52, 54.

Optionally, the flange 53 on the proximal end 52 of the plunger 50 may include one or more passages (not shown) that extend between proximal and distal surfaces of the flange 53. For example, the flange 53 may include a plurality of circular or other enclosed passages spaced apart from one another around a circumference of the flange 53, each extending between the proximal and distal surfaces.

In this option, the flange 53 may be sized and/or shaped to slidably engage an inner wall of the second chamber 24, e.g., to allow the plunger 50 to move from the initial to the extended position, but may not require O-rings or other seals. Instead, one or more O-rings or other seals may be provided within and/or adjacent the inner housing 10 to seal the first and second chambers 22, 24 and/or otherwise a flow path from the gas canister 40 to the plunger 50. For example, a first O-ring 58a may be provided between the inner housing 10 and the opener mechanism 60, a second O-ring 58b may be provided between a distal end 21b of the inner housing 10 and the plunger 50, and/or a third O-ring 58c may be provided between the proximal and distal cylinders 10a, 10b, if provided as separate components secured together.

In one example, the plunger 50 includes a flange 53 formed as a cylindrical head having a larger outer diameter than the plunger 50, which may be integrally molded or otherwise formed with the plunger 50, or that is manufactured separately and permanently attached to the plunger 50, with one or more passages (not shown) extending between proximal and distal surfaces of the flange 53. When the canister 40 is opened to release the pressurized gas, the initial volume that the gas must fill the first chamber 22 around the canister 40, the second chamber 24 around the plunger 50 and, optionally, the plunger chamber 56, which may result in an initial pressure drop as the gas fills the available volume. However, as the plunger 50 advances, the change in volume that the gas must fill increases only minimally (e.g., the volume the plunger 50 occupies within the second chamber 24 that is displaced out of the distal end 16 of drive assembly 12). Consequently, because the volume change is minimized, the resulting force applied by the pressure on the plunger 50 may remain substantially constant or reduce only slightly. Thus, the resulting force drop applied to the plunger 50 may be minimized, which may provide a more uniform delivery rate of the agent from the syringe 70. Additional information regarding plungers that may provide reduced pressure drop can be found in co-pending U.S. application, Ser. No. 17/965,707, the entire disclosure of which is expressly incorporated by reference herein.

Optionally, the proximal end 52 of the plunger 50 may have a larger diameter or other cross-section than the distal end 54 of the plunger 50. For example, the outer diameter or cross-section may taper between the proximal and distal ends 52, 54 of the plunger 50. Such a tapered shape may increase the cross-sectional area of the plunger 50 as it advances from the initial position towards the final position, which may minimize the change in the distal force applied to the syringe stopper due to volume change, which may be particularly useful for applications where consistent rates of delivery are desired. It will be appreciated that any of these optional features related to the plunger 50 may be combined together or omitted, as desired.

Returning to FIGS. 2A and 2B, the opener mechanism 60 includes a spring perch or carriage 64 slidably coupled to the rear housing 12, e.g., within the second and third regions 22b, 22c of the first chamber 24. The carriage 64 carries the opener pin 62, e.g., mounted along the axis 18 and oriented distally towards the cap 44. The carriage 64 and release cap 34 include one or more features that disengage when the release cap 34 is directed proximally (by the activation cap 80 and spacer 30), and the carriage 64 may be biased to move distally, when released, to direct the opener pin distally to open the outlet in the cap 44 of the canister 40.

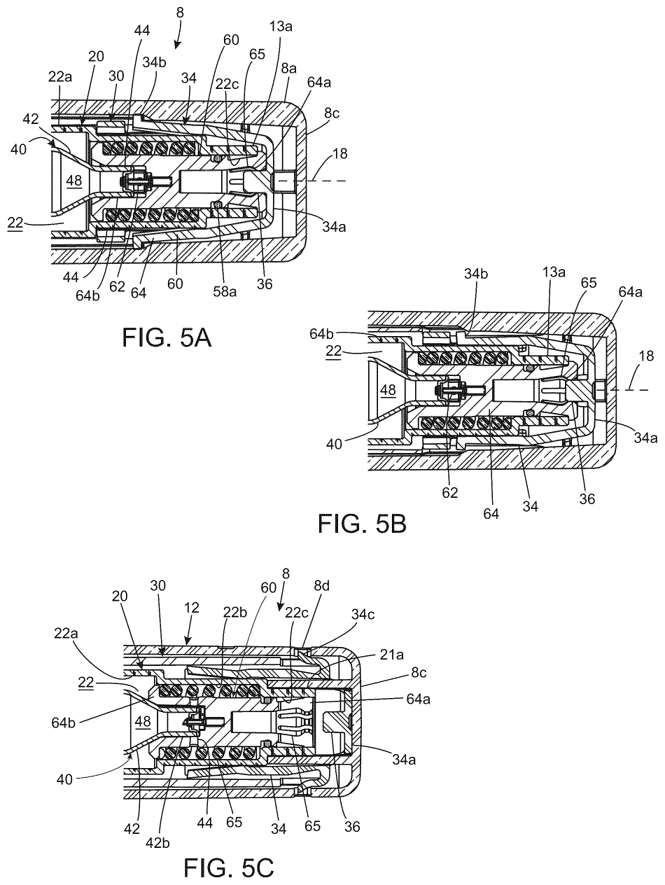

For example, the rear housing 12 and the carriage 64 may include cooperating features that prevent the carriage 64 from moving until the device 6 is activated. For example, as best seen in FIGS. 5A-5C, the carriage 64 includes locking tabs or other features 64a on its proximal end that engage the proximal end 13a of the rear housing 12, and a flange 64b on its distal end slidable within the second region 22b of the first chamber 24. A spring 66 is also provided within the second region 22b around the carriage 64, e.g., a coil spring provided in an initially compressed state between the rear housing 12 and the flange 64b. Consequently, the spring 66 biases the carriage 64 to move distally, but for the locking features 64a engaging the proximal end 13a of the rear housing 12.

The release cap 34 and carriage 64 include cooperating features that release the locking features 64a from the rear housing 12 when the device 6 is activated. For example, as shown in FIGS. 5A-5C, the release cap 34 includes a cylindrical body surrounding a portion of the rear housing 12, e.g., around the second and third regions 22b, 22c of the first chamber 24. The release cap 34 may also include an enclosed proximal end 34a proximal to the proximal end 13a of the rear housing 12, and a distal end 34b that includes one or more features to limit axial movement of the release cap 34. For example, one or more flanges on the distal end 34b may slide proximally along the inner surface of the outer housing 8, e.g., until they abut a ridge on the outer housing 8 to prevent further proximal movement of the release cap 34, as described further elsewhere herein.

As shown, a central hub 36 is provided on the proximal end 34a of the release cap 34 aligned along the axis 18 distally towards the opener pin 62. The carriage 64 includes a plurality of tabs 65, e.g., arranged concentrically around the axis 18 such that the tabs 65 may slide along the hub 36 as the release cap 34 is directed proximally. Once the hub 36 moves proximally beyond the tabs 65, the tabs 65 are biased to move radially inwardly (towards the central axis 18), thereby directing the locking features 64a inwardly. Once the locking features 64a clear the proximal end 13a of the rear housing 12, e.g., as shown in FIG. 5C, the spring 66 may automatically direct the carriage 64 distally, thereby directing the opener pin 62 distally to penetrate the septum in the cap 44 of the canister 40 (or otherwise opening the outlet).

During use, the device 6 may be provided initially with the safety cap 86 attached

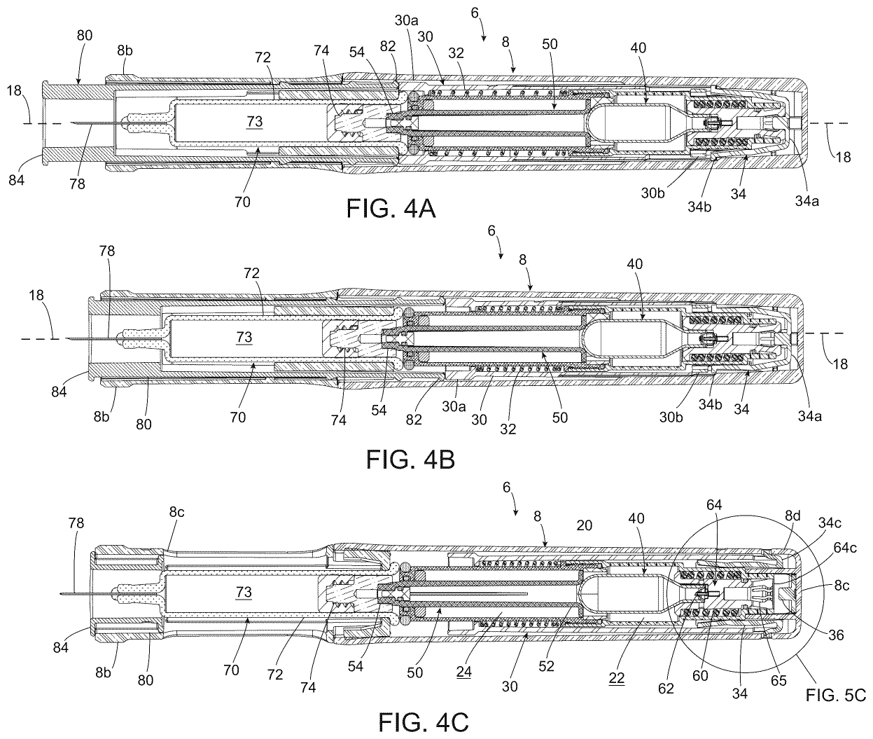

to the distal end 8b of the housing 8, e.g., as shown in FIGS. 1-2B. Immediately before performing an injection, the safety cap 86 may be removed, e.g., by simply pulling the safety cap 86 distally away from the outer housing 8, to expose the contact surface 84 of activation cap 80, e.g., as shown in FIG. 4A. For example, as best seen in FIGS. 3A and 3B, the safety cap 86 may include inner and outer cylindrical members with the proximal end 86a of the inner member coupled to the needle shield 79 and a proximal end 86b of the outer member slidably received over the distal end 8b of the outer housing 8. Alternatively, the safety cap 86 and/or outer housing 8 may include one or more connectors, locks, and the like (not shown) that may be disengaged before removing the safety cap 86.

As described elsewhere herein, the safety cap 86 may prevent the activation cap 80 from being directed proximally, e.g., by preventing the activation cap 80 from being contacted. In addition or alternatively, the safety cap 86 may include features that engage the needle shield 79 such that, when the safety cap 86 is removed, the needle shield 79 is also removed. The safety cap 86 and needle shield 79 may prevent the needle 78 from being exposed from the housing 8 prior to use. Once the safety cap 86 and needle shield 79 are removed, the device 6 is ready to be used to perform an injection.

Turning to FIGS. 4A-4C, the contact surface 84 may be placed against a subject's skin (not shown) and then the device 6 may be pressed against the skin to insert the needle 78, thereby causing the activation cap 80 to slide proximally from the initial position shown in FIG. 4A to the retracted position shown in FIG. 4B as the needle 78 enters the subject's skin. As described further below, this action may activate the opener mechanism 60 to cause the opener pin 62 to open the outlet of the gas canister 40 to release pressurized gas into the first chamber 22, whereupon the pressurized has may advance the plunger 50 and, consequently, the stopper 74 to deliver the agent(s) through the needle 78 into the subject.

For example, as shown in FIG. 4A, initially, the proximal end 82 of the activation cap 80 may abut or otherwise engage a distal end 30a of the spacer 30, and a proximal end 30b of the spacer 30 may abut or otherwise engage the distal end 34b of the release cap 34. Consequently, when the activation cap 80 is retracted proximally within the outer housing 8, its proximal end 82 pushes the distal end 30a of the spacer 30, thereby directing the spacer 30 proximally, and, in turn, the proximal end 30b of the spacer 30 pushes the distal end 34b of the release cap 34, thereby directing the release cap 34 proximally, e.g., until the proximal end 34a of the release cap 34 contacts the proximal end 8c of the outer housing 8.

As the activation cap 80, spacer 30, and release cap 34 move proximally, the inner housing 20 (and canister 40 within the first chamber 22) may remain substantially stationary. As can be seen in FIGS. 4A and 4B, as the spacer 30 is directed proximally, the spring 32 may be compressed, e.g., between features on the distal end 30a of the spacer 30 and the inner housing 20, thereby biasing the spacer 30 subsequently to move distally. However, with the operator pressing the contact surface 84 of the activation cap 80 against the subject's skin, the spacer 30 is unable to move distally.

Since the carriage 64 is secured relative to the inner housing 20 by the locking features 64a, the carriage 64 also remains substantially stationary, such that the release cap 34 moves proximally relative to the carriage 64 and the tabs 65 on the carriage 64 slide along the hub 36 on the release cap 34, as best seen in FIGS. 5A-5C. When the release cap 34 reaches its proximal-most position, e.g., shown in FIGS. 4C and 5C, the hub 36 has moved proximally beyond the ends of the tabs 65 and, given the inward bias of the tabs 65, the tabs 65 are released and automatically move inwardly. Given that the tabs 65 are coupled to the locking features 64a on the carriage 64, the inward movement of the tabs 65 causes the locking features 64a to move inwardly, thereby disengaging the locking features 64a from the proximal end 21a of the inner housing 20.

Due to the spring 66, the carriage 64 is then released and biased to move distally, thereby advancing the opener pin 62 distally to open the outlet of the canister 40, as shown in FIGS. 4C and 5C. The release cap 34 may include one or more tabs or other features 34c, e.g., corresponding to the pockets 8d in the outer housing 8, such that, when the release cap 34 reaches its distal-most position, the tabs 34c are received in respective pockets 8d, as shown. Thus, the tabs 34c may prevent the release cap 34 from moving distally as the carriage 64 advances distally due to the spring 66.

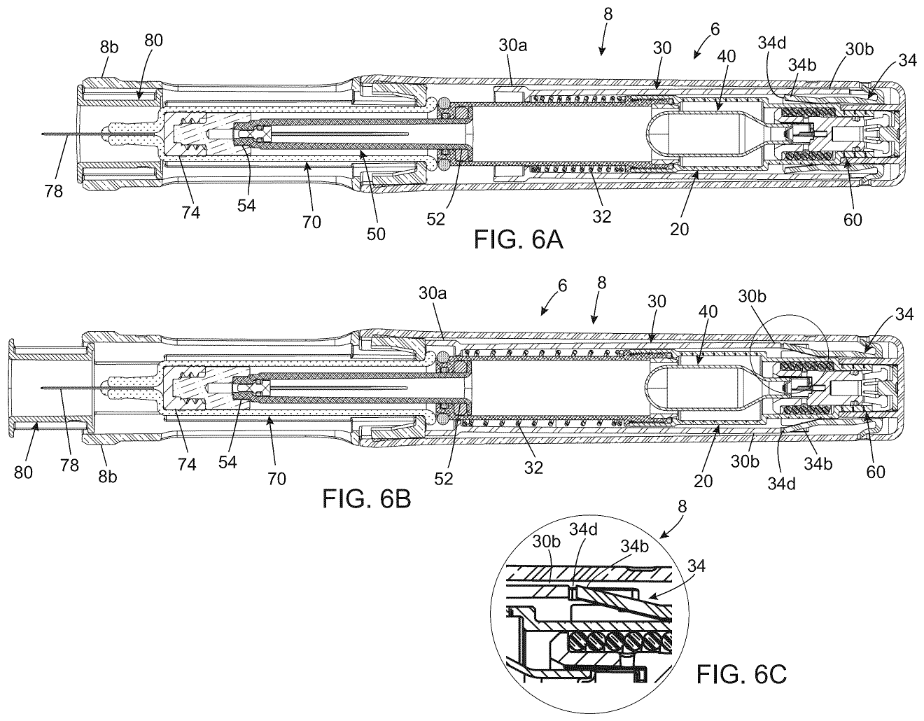

When the carriage 64 moves distally, it causes the opener pin 62 to advance and penetrate the septum in the cap 44 (or otherwise open the outlet), thereby releasing the pressurized gas within the canister 40. The released pressurized gas then enters the first chamber 22, e.g., around the canister 40, and passes to the second chamber 24, thereby pressurizing the second chamber 24 to generate a distal force to direct the plunger 50 distally from the initial position towards a final position to deliver the one or more agents from the syringe 70 through the needle 78 into the subject, e.g., as shown in FIG. 6A.

The operator may monitor the syringe 70 during the injection, e.g., via the window(s) 8c to confirm that the stopper 74 has fully advanced distally within the barrel 72 to deliver the entire dose into the subject. Once the injection is completed, the device 6 may be directed away from the subject, thereby withdrawing the needle 78 from the subject's skin. As the device 6 is directed away from the subject's skin, the activation cap 80 is free to move distally and, due to the distal bias of the spring 32, the spacer 30 automatically advances distally, thereby directing the activation cap 80 distally to cover the needle 78 as it is withdrawn.

As best seen in FIG. 6C, as the spacer 30 advances distally, the proximal end 30b of the spacer may pass distally beyond features 34d on the distal end 34b of the release cap 34. For example, the features 34b may include one or more fingers or tabs that slide along the interior of the spacer 30 until the proximal end 30b passes distally, whereupon the tabs may resiliently move radially outwardly, e.g., such that the tabs 34d are located proximal to the proximal end 30b of the spacer 30, thereby preventing the spacer 30 from subsequently moving proximally. With the spacer 30 prevented from moving proximally, the activation cap 80 is unable to retract again, thereby locking the activation cap 80 extended over the needle 78 and preventing accidental exposure of the needle 78.

If the device 6 is a single-use device, the device 6 may then be disposed of. If the device is reusable, e.g., if the forward and rear housings 10, 12 are separable, the rear housing 12 may subsequently be separated from the forward housing 10, and the syringe 70 may be removed. For a reusable device, one or more features may be provided within the device to release any residual pressure within the first and second chambers 22, 24. For example, as shown in FIGS. 6A and 6B, a gas valve and/or shaft member may be provided within the plunger 50, which may be manipulated to release the pressurized gas before removing decoupling the plunger 50 from the stopper 74. The separate housings 10, 12 may then be cleaned and/or sterilized and a new gas canister 40 may be loaded into the inner housing 20 before reassembling the device.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A method for assembling an injector device, comprising:
providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end;
providing a rear housing comprising proximal and distal ends, a source of pressurized gas within the rear housing, and a plunger including a first end within a chamber of the rear housing such that gas released from the source travels to the chamber to direct the plunger distally;
with a second end of the plunger extending from the distal end of the rear chamber, aligning the proximal end of the forward housing with the distal end of the rear housing such that the second end of the plunger is adjacent the stopper; and
directing the proximal end of the forward housing towards the distal end of the rear housing, thereby causing the plunger to retract proximally within the rear housing to a proximal position.

2. The method of claim 1, wherein the proximal end of the forward housing is directed towards the distal end of the rear housing until connectors engage to secure the forward and rear housings together.

3. The method of claim 2, wherein, when the connectors engage to secure the forward and rear housings together, the plunger is in a proximal-most position without a substantial gap between the second end of the plunger and the syringe stopper such that, when pressurized gas is released from the source of pressurized gas when the injector device is subsequently activated, the pressurized gas advances the plunger towards a distal position and immediately applies a distal force to the stopper.

4. The method of claim 2, wherein the stopper does not move relative to the syringe when the plunger is retracted proximally such that the stopper directs the plunger proximally when the proximal end of the forward housing is directed towards the distal end of the rear housing.

5. The method of claim 2, wherein the forward housing comprises an activation cap on the distal end such that a contact surface is disposed distal to the distal end of the forward housing and the rear housing comprises a release cap carrying an opener pin located adjacent the source of pressurized gas that is operatively coupled to the activation cap when the connectors engage such that, when the contact surface of the activation cap is pressed against a subject's skin, the activation cap moves proximally to direct the release cap to cause the opener pin to open the source of pressurized gas to release the pressurized gas into the first chamber and into the second chamber such that the pressurized gas advances the plunger to direct the stopper distally to deliver the one or more agents from the outlet.

6. The method of claim 5, wherein the syringe comprises a needle communicating with the outlet and positioned adjacent the distal end of the forward housing.

7. The method of claim 1, wherein the plunger is positioned in a distal-most position extending from rear housing before the forward and rear housings are aligned with and directed towards one another.

8. The method of claim 7, wherein a flange on the proximal end of the plunger contacts a distal wall of the chamber in the distal-most position.

9. The method of claim 1, further comprising a piston member on the stopper that contacts the distal end of the plunger directing the proximal end of the forward housing towards the distal end of the rear housing, thereby causing the plunger to retract proximally within the rear housing to the proximal position.

10. A method for assembling an injector device, comprising:
providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end;
providing a rear housing comprising proximal and distal ends, a source of pressurized gas within the first chamber, and a plunger including a first end within a chamber of the rear housing such that gas released from the source travels to the chamber to direct the plunger distally;
with a second end of the plunger extending from the distal end of the rear housing in a distal-most position, aligning the proximal end of the forward housing with the distal end of the rear housing such that the second end of the plunger is adjacent the stopper;
directing the proximal end of the forward housing towards the distal end of the rear housing, thereby causing the plunger to retract proximally within the rear housing to a proximal position; and
coupling one or more connectors to secure the forward and rear housings together, the plunger being positioned in a proximal-most position.

11. A device for delivering one or more agents into a subject's body, comprising:
an outer housing comprising a proximal portion terminating in a proximal end and a distal portion terminating in an open distal end;
a syringe comprising a barrel containing one or more agents, a needle extending distally from the barrel such that a tip of the needle is disposed within the distal portion adjacent the distal end, and a stopper within a proximal end of the barrel;
an inner housing within the outer housing comprising one or more chambers therein;
an activation cap on the distal end of the housing such that a contact surface is disposed distal to the outer housing distal end;
a source of pressurized gas within the inner housing;
a release cap within the outer housing carrying an opener pin located adjacent the source of pressurized gas; and
a plunger comprising a proximal end within a chamber of the one or more chambers and a distal end positioned adjacent and spaced apart proximally from the stopper by a gap distance;
the activation cap operably coupled to the release cap and movable axially relative to the outer housing such that, when the contact surface of the activation cap is pressed against a subject's skin, the activation cap is configured to move proximally to direct the release cap to cause the opener pin to open the source of pressurized gas to release the pressurized gas into the chamber such that the pressurized gas advances the plunger to direct the distal end of the piston distally into contact with the stopper to advance the stopper to deliver the one or more agents through the needle.

12. The device of claim 11, wherein the proximal portion comprises a rear housing carrying the inner housing, the release cap, the source of pressurized gas, and the plunger, the plunger positioned adjacent a distal end of the rear housing.

13. The device of claim 12, wherein:
the distal portion comprises a forward housing carrying the activation cap and including a proximal end comprising an opening for inserting the syringe into an interior of the forward housing; and
the proximal end of the forward housing and the distal end of the rear housing comprise cooperating connectors for securing the forward and rear housings together after inserting the syringe to provide the outer housing.

14. The device of claim 13, wherein, when the forward and rear housings are secured together, the distal end of the plunger is spaced apart proximally from the stopper by the gap distance.

15. The device of claim 11, wherein the source of pressurized gas comprises a canister including an outlet, and wherein the opener pin is movable axially to open the outlet to release the pressurized gas.

16. The device of claim 15, wherein the gas canister comprises a penetrable septum initially spaced away from the opener pin and, wherein, when the release cap is directed proximally, the opener pin is released such that the opener pin resiliently moves axially to penetrate the septum and release the pressurized gas.

17. The device of claim 11, further comprising a carriage releasably coupled to the release cap, the opener pin extending axially from the carriage, the release cap and carriage comprising one or more features that disengage when the release cap is directed proximally by the activation cap, the carriage biased to move axially when released to move the opener pin to open the outlet.

18. A method for assembling an injector device, comprising:
providing a forward housing comprising proximal and distal ends and an interior, a syringe loaded into the interior such that a stopper of the syringe is positioned adjacent the proximal end, the stopper slidable distally within the syringe to deliver one or more agents from an outlet of the syringe adjacent the distal end;
providing a rear housing comprising proximal and distal ends, a source of pressurized gas within the rear housing, and a plunger including a proximal end within a chamber of the rear housing such that gas released from the source travels to the chamber to direct the plunger distally; and
with the plunger in an initial position proximal to a distal-most position within the chamber, coupling the proximal end of the forward housing to the distal end of the rear housing such that a distal end of the plunger is spaced apart proximally from the stopper.

19. The method of claim 18, wherein the proximal end of the forward housing is coupled to the distal end of the rear housing by one or more connectors.

20. The method of claim 18, wherein, the plunger is provided in a proximal-most position before coupling the proximal end of the forward housing to the distal end of the rear housing.

21-26. (canceled)
